(19) United States
(12) Patent Application Publication
(10) Pub. No.: US 2024/0139270 A1
(43) Pub. Date: May 2, 2024

(54) COMPOSITIONS

(71) Applicant: Givaudan SA, VERNIER (CH)

(72) Inventors: Pascale Elizabeth Renèe FANÇA-BERTHON, Le Thor (FR); Mathieu TENON, Malemort du Comtat (FR); Laure ZIEGLER, Pernes les Fontaines (FR)

(21) Appl. No.: 18/278,731

(22) PCT Filed: Feb. 23, 2022
(86) PCT No.: PCT/EP2022/054489

§ 371 Date: Aug. 24, 2023

(30) Foreign Application Priority Data
Feb. 25, 2021 (GB) 2102704.0

(51) Int. Cl. A61K 36/53; A61K 31/192; A61K 31/216; A61K 31/366; A61K 36/74; A61P 9/10

(52) U.S. Cl. CPC A61K 36/53; A61K 31/192; A61K 31/216; A61K 31/366; A61K 36/74; A61P 9/10; A61K 2236/331

(57) ABSTRACT

The present invention relates to compositions comprising a green coffee extract and a Lamiaceae extract (such as a rosemary extract), the use of these composition in treating cardiovascular conditions, in particular, cardiovascular conditions resulting from atherosclerosis and methods of making the compositions.

The present invention relates to compositions comprising a green coffee extract and a Lamiaceae extract (such as a rosemary extract), the use of these composition in treating cardiovascular conditions, in particular, cardiovascular conditions resulting from atherosclerosis and methods of making the compositions.

The present invention also relates to pharmaceutical formulations, nutraceutical formulations and food products comprising the composition.

The listing or discussion of an apparently prior-published document in this specification should not necessarily be taken as an acknowledgement that the document is part of the state of the art or is common general knowledge.

Atherosclerosis represents the major cause of death and cardiovascular morbidity in the western world.

Atherosclerotic vasculopathy is a multifactorial process causing vessels damage and cardiovascular diseases, the leading causes of death worldwide. Atherosclerotic plaque is the asymptomatic primary, elementary, lesion of atherosclerotic vasculopathy.

Accumulation of the oxidized low-density lipoprotein (oxLDL) at sub endothelial sites is now recognized as one of the major trigger events in plaque formation

Risk factors for atherosclerosis include high oxidized low-density lipoprotein (LDL) cholesterol levels, low high-density lipoprotein (HDL) cholesterol levels, hypertension, diabetes mellitus, family history, male gender, cigarette smoke, and high serum cholesterol.

Oxidized low-density lipoprotein (LDL) is one of the most relevant risk factors for atherosclerosis plaques formation. Accumulation of the oxidized low-density lipoprotein (oxLDL) at sub endothelial sites is now recognized as one of the major trigger events in plaque formation (C. F. Suciu et al. Autoimmunity Reviews Volume 17, Issue 4, April 2018, Pages 366-375)

Also 8-isoprostaglandin F2α (8-isoPGF2α, also known as 8-epi-PGF2α or 8-isoprostane) is the most commonly used biomarker for the assessment of oxidative stress (Ito, Fumiaki et al. Antioxidants (Basel, Switzerland) vol. 8,3 72. 25 Mar. 2019, doi:10.3390/antiox8030072)

The use of synthetic or artificial ingredients within health/pharmaceutical products has become a concern due to the possible negative effects they may have on consumers' health and care.

This has resulted in an increasing public demand for natural alternatives to artificial ingredients in health/pharmaceutical/cosmetic products.

Therefore, it is of interest to provide natural (non-synthetic) compositions that can be used against cardiovascular diseases, in particular those resulting from atherosclerosis.

It has been surprisingly and unexpectedly found by the present inventors that a combination comprising a green coffee extract and a Lamiaceae extract can reduce the formation of atherosclerotic plaques, reduce LDL, increase HDL, and decrease F_{2α}-isoprostane (such as 8-iso-PGF2α) levels.

Thus, the present invention provides a combination comprising an extract obtained or obtainable from a plant of the Coffea genus and a plant of the Lamiaceae family. This combination is hereinafter referred to as the combination of the invention.

The plants of the Coffea genus include Coffea canephora, Coffea arabica (Arabica), Coffea canephora (Robusta), Coffea liberica (Liberica), etc.

Typically, the extract obtained from a plant of the Coffea genus may be obtained from the beans of the plant, for example the green beans.

The plants of the Lamiaceae family include Salvia (such as Salvia apiana and Salvia officinalis), Rosmarinus (such as Rosmarinus officinalis), Lepechinia, Oreganum, Thymus, Hyssopus and mixtures thereof. For example, rosemary, sage, oregano, thyme, mints and mixtures thereof.

Typically, the extract obtained from a plant of the Lamiaceae family may be obtained from the aboveground parts, such as the leaves of the plant.

In the combination of the invention, the extract obtained or obtainable from a plant of the Coffea genus may be present in the combination in an amount of from about 1% to about 99%, such as from about 65% to about 80% by weight of the combination (such as from about 70% to about 75% by weight of the combination) and the extract obtained or obtainable from a plant of the Lamiaceae family may present in the combination in an amount of from about 1% to about 99%, such as from about 20% to about 35% by weight of the combination (such as from about 25% to about 30% by weight of the combination).

The extract obtained from a plant of the Coffea genus and/or a plant of the Lamiaceae family may be an aqueous extract, an alcohol extract (which includes hydro-alcoholic extracts) or an organic extract.

The term “aqueous extract” as used herein, refers to the extract obtained from a plant of the Coffea genus and/or a plant of the Lamiaceae family when the extraction from the plant has been performed using water as the only solvent.

The term “alcohol extract” as used herein, refers to the extract obtained from a plant of the Coffea genus and/or a plant of the Lamiaceae family when the extraction from the plant has been performed using an alcohol as the solvent. The alcohol solvent may consist of only alcohol (e.g. 100% alcohol), for example 100% ethanol, or may be a mixture of an alcohol and water (i.e. a hydro-alcoholic solvent), for example, a mix of ethanol and water (hydro-ethanolic solvent), for example, from about 1% to about 99% alcohol (e.g. ethanol) in water, for example the ratio of water to alcohol is from 10/90% v/v to 90/10% v/v or 30/70% v/v to 70/30% v/v, such as 50/50% v/v or 70/30 v/v.

The term “organic extract” as used herein, refers to the extract obtained from a plant of the Coffea genus and/or a plant of the Lamiaceae family when the extraction has been performed using an organic solvent that is not an alcohol. For example, the organic solvent may be selected from the group consisting of acetic acid, acetone, acetonitrile, benzene, 2-butanone, carbon tetrachloride, chlorobenzene, chloroform, cyclohexane, 1,2-dichloroethane, diethylene glycol, diethyl ether, diglyme (diethylene glycol, dimethyl ether), 1,2-dimethoxy-ethane (glyme, DME), dimethyl-formamide (DMF), dimethyl sulfoxide (DMSO), 1,4-dioxane, ethyl acetate, ethylene glycol, glycerin, heptane, hexamethylphosphoramide (HMPA), hexamethylphosphorous, triamide (HMPT), hexane, methyl t-butyl, ether (MTBE), methylene chloride, N-methyl-2-pyrrolidinone (NMP), nitromethane, pentane, petroleum ether (ligroine), pyridine, tetrahydrofuran (THF), toluene, triethyl amine, o-xylene, m-xylene and p-xylene.

In a preferred aspect, the extract obtained from a plant of the Coffea genus is an alcoholic extract. In particular, a hydro-alcoholic extract, such as a hydro-ethanolic extract (i.e. an extract obtained using 70% water:30% ethanol).

In a preferred aspect, the extract obtained from a plant of the Lamiaceae family is an alcoholic extract, i.e. an extract obtained using 100% ethanol.

As will be appreciated by the person skilled in the art, as used herein the term “obtainable from” means that the extract may be obtained from a plant or may be isolated from the plant, or may be obtained from an alternative source, for example by chemical synthesis or enzymatic production. Whereas the term “obtained” as used herein, means that the extract is directly derived from the plant source.

The extract obtained from a plant of the Coffea genus may comprise chlorogenic acid in an amount of about 20% or greater by weight of the extract, such as about 30% or about 40% or greater. For example, the amount of chlorogenic acid present in the extract may be from about 20%, 30% to about 60% by weight of the extract, such as from about 45% to about 50% by weight of the extract.

The extract obtained from a plant of the Lamiaceae family may be enriched in phenolic diterpenes such as carnosic acid, carnosol, methylcarnosate, other phenolic diterpene derivatives such as rosmanol, isorosmanol, 1 1, 12-di-O-methylisorosmanol, 12-O-methylcarnosic acid, rosmanol-9-ethyl ether, circimaritin, Methylated monooxidized product of carnosic acid, genkwanin, epirosmanol, epiisorosmanol, carnosic acid derivative, epirosmanol ethyl ether, cryptotanshinone and mixtures thereof.

The extract obtained from a plant of the Lamiaceae family may comprise at least about 1%, at least about 2%, at least about 3%, at least about 4%, at least about 5%, at least about 6%, at least about 7%, at least about 8%, at least about 9%, at least about 10%, at least about 15%, at least about 20%, at least about 30%, at least about 40%, at least about 50%, at least about 60%, at least about 70%, at least about 80%, at least about 90%, at least about 95%, or at least about 99 wt % of one or more phenolic diterpenes such as those described above. Such as from about 1% to about 99 wt % or from about 10% to about 90 wt % or from about 20% to about 80 wt % of one or more phenolic diterpenes.

For example, the extract obtained from a plant of the Lamiaceae family may comprise carnosic acid and/or carnosol in the amounts defined above.

The extract obtained or obtainable from a plant of the Lamiaceae family may comprise from about 15% to about 30% by weight of the extract of carnosic acid, such as from about 20% to about 25% by weight of the extract.

The extract obtained or obtainable from a plant of the Lamiaceae family may comprise from about 1% to about 5% by weight of the extract of carnosol acid.

The extract obtained from a plant of the Lamiaceae family (such as rosemary and/or Salvia extract) may comprise (or consist essentially/consist of):
a) from about 1%, 5%, 10%, 15%, 20%, 25%, 30%, 35%, 40% or 45% to about 95%, 90%, 85%, 80%, 75%, 70%, 65%, 60%, 55% or 50% by weight of the final composition (w/w) of carnosic acid, such as from about 20% to about 80% w/w, preferably such as from about 30% to about 50% w/w such as from about 15% w/w to about 30% wt/wt such as about 20% wt/wt and/or
b) from about 1%, 5%, 10%, 15%, 20%, 25%, 30%, 35%, 40% or 45% to about 95%, 90%, 85%, 80%, 75%, 70%, 65%, 60%, 55% or 50% of carnosol by weight of the final composition (w/w), such as from about 1% to 20% w/w such as from about 2% to about 10% w/w, preferably such as from about 2% to about 5% w/w, such as about 2.28% w/w.

The extract obtained from a plant of the Lamiaceae family (such as rosemary and/or Salvia extract) may comprise (or consist essentially/consist of) the following phenolic diterpenes:carnosic acid and/or carnosol and the ratio between carnosic acid and carnosol is from 40:1 to 1:40, such as 30:1, 20:1, 10:1, 5:1 or 1:1, In a preferred embodiment the ratio is of about 10:1.

In some aspects, the extract obtained from a plant of the Lamiaceae family (such as rosemary and/or Salvia extract) may be:
substantially free of other plant material (e.g. free of plant cellulose);
substantially free of plant cells; and/or
substantially free of plant cellular matter,
substantially free of toxic components like pesticides, quintozene, aflatoxins, ochratoxin A, cadmium, arsenic, lead or mercury,
substantially free of solvents
substantially free of volatile oil components.

By the term “substantially free”, we mean that the extract obtained from the plant of the Lamiaceae family comprises less than about 5% by weight of the extract of the listed components, such as less than about 2% or less than about 1% or 0.1% by weight of the extract.

The extract obtained from a plant of the Lamiacea family may have a majority of the volatile oil components removed.

As used herein, the term “volatile oil components” may refer to compounds like essential oils such as: (−)-borneol, (−)-bornyl acetate, (−)-camphor, 1,8-Cineole (eucalyptol) and verbenone.

For example, the ration between the total % of phenolic diterpenes (such as carnosic acid and carnosol)/Total % of volatiles oil components (such as (−)-borneol, (−)-bornyl acetate, (−)-camphor, 1,8-Cineole (eucalyptol) and verbenone) may be not less than 15.

Thus, the present invention also provides a mixture comprising
a) from about 19.5% to about 50% wt/wt, such as from about 30% to about 40% wt/wt, such as about 35% by weight of the composition of chlorogenic acid(s),
b) from about 3% to about 15% wt/wt, such as from about 3% to about 8%, such as about 5% wt/wt by weight of the composition of carnosic acid, and
c) from about 0.2% to about 3% wt/wt, such as from about 0.4% to about 1% such as 0.6% wt/wt by weight of the composition of carnosol.

In the mixture of the invention, the chlorogenic acid(s) or CGA(s) may be obtained or obtainable from an extract from a plant of the Coffea genus, such as an aqueous or alcoholic extract as defined previously. The mixture of the invention may comprise one or more than one CGAs. Alternatively, the chlorogenic acid(s) may be obtained from other biological material that comprises chlorogenic acid (CGA) (also referred to herein after as chlorogenic acid(s) containing biological material) by synthetic means, such as via chemical synthesis.

The mixture of the invention can further contain caffeic acid. Caffeic acid may be obtained or obtainable from an extract from a plant of the Coffea genus, such as an aqueous or alcoholic extract as defined previously. Alternatively, the caffeic acid may be obtained from other biological material that comprises caffeic acid or may be obtained by synthetic means, such as via chemical synthesis.

In the mixture of the invention, the carnosic acid may be obtained or obtainable from an extract from a plant of the Lamiaceae family, such as an aqueous or alcoholic extract as defined previously. Alternatively, the carnosic acid may be obtained from other biological material that comprises carnosic acid or may be obtained by synthetic means, such as via chemical synthesis.

In the mixture of the invention, the carnosol may be obtained or obtainable from an extract from a plant of the Lamiaceae family, such as an aqueous or alcoholic extract as defined previously. Alternatively, the carnosol may be obtained from other biological material that comprises carnosol or may be obtained by synthetic means, such a via chemical synthesis.

“Biological material” refers to any material that has been obtained from or is obtainable from plants (plant biological material), animals (animal biological material) or prokaryote (prokaryotic biological material).

As used herein, the term “plant biological material” is material that has been obtained from or is obtainable from plants (including algae), such as roots and/or the aerial parts of the plant, such as leaves, flowers, stems, barks, fruits or seeds or their tissues. For example, the plant biological material may be obtained from the fruits of the plant. Plant biological material includes also residues from agricultural harvesting and industrial processing of said materials.

As used herein, the term “animal biological material” is material that has been obtained from or is obtainable from an animal source, such as from secretions from the glands of mammals.

As used herein, the term “prokaryotic biological material” is material that has been obtained from or is obtainable from single cell organisms, such as bacteria.

“Chlorogenic acid(s) containing biological material” means that the biological extract com-prises about 0.05% or more by weight of one or more chlorogenic acid(s).

For example, the CGA(s) containing biological material may comprise about 0.5% or more, 2% or more, about 5% or more, about 10% or more, about 20% or more, or about 40% or more by weight of CGA(s).

The CGA(s) containing biological material is preferably plant biological material. The plant biological material may be obtained from or obtainable from plant roots and/or plant aerial parts, such as the leaves, flowers, stems, barks, fruits and/or seeds, their tissues (such as the rind of the fruit) or mixtures thereof. For example, the plant biological material may be the leaves of the plant.

Non-limitative examples of CGA(s) containing plant biological material are green coffee beans from any species of the genus Coffea such as Coffea arabica (Arabica), Coffea canephora (Robusta), Coffea liberica (Liberica), etc; leaves of Ilex paraguariensis, pome fruits (e.g., apples and pears), stone fruits (e.g., cherries and plums), berry fruits, citrus fruits, Brassica vegetables (e.g., kale, cabbage and brussel sprouts), solanaceae (e.g., potato tubers, tomatoes, and aubergines), asteraceae (e.g., chicoryroot and artichokes), and a variety of other miscellaneous vegetables. It may also be found in cereal grains (e.g., oats, barley, rye, rice, corn and wheat). The amount and different types of chlorogenic acid that are present vary depending upon the source. Chlorogenic acid may be extracted from one or more botanical sources, and/or synthetic chlorogenic acid may be used.

The CGA(s) containing plant biological material may be Arabica, Robusta and/or Liberica green coffee beans.

The CGA(s) of natural origin can be present in the composition of the invention as purified CGA(s) or as natural extracts obtained or obtainable from any of the CGA(s) containing biological material (such as Arabica, Robusta and/or Liberica green coffee beans) mentioned before.

Typically, the extracts comprising CGA(s) extracted from a Chlorogenic acid(s) containing biological material may have a total chlorogenic Acids (TCGA content) of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40% to about 99, 90, 80, 70, 60, 50% wt/wt, such as 40 to 50% wt/wt TCGA.

As used herein, the term “TCGA content” means the Total Chlorogenic Acids (TCGA) content, which is calculated as the sum of the concentrations of one of more of the CGA(s) mentioned before in a wt/wt dry basis.

Other components can be also presented such as terpenes, phenolic compounds, amino acids, flavonoids, volatile oils, vitamins, and minerals.

Where the biological material is Arabica, Robusta and/or Liberica green coffee beans and the natural extracts comprising CGA(s)” (such a concentrate or a dry form) may comprise from about 5% to about 99%, from about 5% to about 40%, from about 10% to about 35%, more precisely about 33% of solids and a TCGA content from 3 to 50% by weight of the extract.

This natural extract can be used for the formulation of the composition or the mixture of the invention.

However, after completion of the extraction process, the CGA(s) may be isolated from the extract (i.e. purified) using suitable purification processes, such as a chromatographic process.

For example, purified CGA(s) may be obtained using the following process:
the natural source containing CGA(s) such as Arabica, Robusta and/or Liberica green coffee beans powder (i.e. obtained by preparing ground Arabica, Robusta and/or Liberica green coffee beans) is dissolved in an alcohol and the CGA(s) are extracted by alcohol (such as methanol) from the powder.
The alcohol is then evaporated and the remaining residue including CGA(s) is loaded into a chromatography column filled with reverse-phase C-18 resin;
several fractions containing different compounds are eluted with a series of water and 10% MeOH/90% water, and MeOH system. The fractions are compared by high performance liquid chromatography (HPLC) analysis and those elutes having similar HPLC patterns are combined;
the combined fractions are separated on normal phase silica gel column chromatography and eluted with chloroform (CHCl3), CHC
methanol mixture starting from 90%, 80% CHCl3 to 100% MeOH to give several sub-fractions. The sub-fractions are compared by HPLC and the fractions which contain CGA(s) are combined, respectively. The combined fractions are further purified by a combination of column chromatography over C-18, MCI GEL CHP-20P and/or Sephadex LH-20 resins to provide pure CGA(s).

The terms “isolated” and “purified” as used herein refer to the extract or CGA(s) being separated from at least one other component (e.g. terpenes, phenolic compounds, amino acids, flavonoids, volatile oils, vitamins, minerals etc) present with the extract or CGA(s) in its natural source. For example, the extract or CGA(s) may be provided in pure form or in the presence of a solvent, buffer, ion, or other component normally present in a solution of the same. Typically, the purification results in the content of CGA(s) being more 60% or more by weight of the extract, such as 70% or more, 80% or more or 99% or more.

The composition of the invention may have a TCGA content of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40% to about 99, 90, 80, 70, 60, 50% wt/wt. The at least one chlorogenic acid concentration (or TCGA content) may be from about 10 to about 50%, more precisely from about 30 to about 50% wt/wt (in relation with the total weight of the composition) such as from 40% to 50%, such as 35% wt/wt.

Typically, as previously described, the CGA(s) are of natural origin obtained or obtainable from a CGA(s) containing biological material (such as Arabica, Robusta and/or Liberica green coffee beans) and may comprises neo-chlorogenic acid (neo-CGA; 5-O-caffeoylquinic acid or 5-CQA), crypto-chlorogenic acid (crypto-CGA; 4-O-caffeoylquinic acid or 4-CQA), n-chlorogenic acid (n-CGA; 3-O-caffeoyl quinic acid or 3-CQA), iso-chlorogenic acid A (iso-CGA A; 3,5-dicaffeoylquinic acid) iso-chlorogenic acid B (iso-CGA B; 3,4-dicaffeoylquinic acid), iso-chlorogenic acid C (iso-CGA C; 4,5-dicaffeoylquinic acid, 4.5-di-QA), 3,5-dicaffeoylquinic acid (3.5-di CQA), 3,4-Caffeoylquinic acid (of 3.4-CFQA), 3,5-Caffeoylquinic acid (3.4-CFQA), 4,5-Caffeoylquinic acid (4.5-CFQA), feruloylquinic acids (3-FQA, 4-FQA, and 5-FQA) or any combination of any of said CGA(s) or other CGA(s) described on the literature, and mixtures thereof.

The composition of the invention may have about 8% of 3-CQA, about 13.2% of 5-CGA (5-CQA), about 2% of 3-FQA, about 9.4% of 4-CQA, about 3.3% of 4-FQA, about 2.3% of 5FQA, about 2.44 of 3.4-di-CQA, about 1.-6% of 3.5-di-CQA, about 3.8% e⁻² of 4.5-di-QA, about 3.1% of 3.4-CFQA, about 0.4% of 3.5-CFQA, about 0.9% of 4.5-CFQA, about 0.5% of C-try and/or about 8.2 e⁻²% of p-coumatryl-tryp and mixtures thereof.

The composition of the invention may comprise:
from about 5% to about 15%, such as from 7% to 10%, such as 8% of 3-CQA, and/or
from about 5% to about 20%, such as from about 10% to about 15%, such as 13.2% of 5-CGA (5-CQA), and/or
from about 0.5% to about 5%, such as from about 1% to 3%, such as about 2% of 3-FQA, and/or
from about 5% to about 15%, such as from about 7% to about 10%, such as 9.4% of 4-CQA, and/or
from about 1% to about 6%, such as from 2% to about 4%, such as 3.3% of 4-FQA, and/or
from about 0.5% to about 6%, such as from 1% to about 4%, such as 2.3% of 5FQA, and/or
from about 0.5% to about 6%, such as from 1% to about 4%, such as about 2.44 of 3.4-di-CQA, and/or
from about 0.5% to about 4%, such as from 1% to about 2%, such as about 1.6% of 3.5-di-CQA, and/or
from about 0.5% to about 6%, such as from 1% to about 4%, such as about 3.8% e⁻² of 4.5-di-QA, and/or
from about 0.5% to about 6%, such as from 1% to about 4%, such as about 3.1% of 3.4-CFQA, and/or
from about 0.1% to about 2%, such as from 0.2% to about 1%, such as about 0.4% of 3.5-CFQA, and/or
from about 0.1% to about 2%, such as from 0.2% to about 1%, such as about 0.9% of 4.5-CFQA, and/or
from about 0.1% to about 2%, such as from 0.2% to about 1%, such as about 0.5% of C-try and/or
from about 1% to about 10%, such as from 5% to about 9%, such as about 8.2 e⁻²% of p-coumatryl-tryp.

In one embodiment of the combinations or mixtures defined herein, the 5-caffeoylquinic acid/total chlorogenic acids ratio is from 0.2 to about 0.3.

In any of the combinations or mixtures defined herein, the extract obtained or obtainable from a plant of the Coffea genus and/or the extract of a plant of the lamiaceae family may further comprise maltodextrin. Maltodextrins are commonly used as excipients or carriers.

Maltodextrins are defined as starch hydrolysis products with dextrose equivalent less than 20. Dextrose equivalent (DE value) is a measure of the reducing power of starch derived oligosaccharides expressed as percentage of D-glucose on dry matter of hydrolysate and is inverse value of average degree of polymerisation (DP) of anhydro glucose units. As products of starch hydrolysis, maltodextrins contain linear amylose and branched amylopectin degradation products, therefore they are considered as D-glucose polymers joined by a-(1,4) and a-(1,6) linkages.

Although maltodextrins are derived from a natural compound (starch), their structure is different from the initial structure of the natural molecule they derive from (starch). This difference is induced by the hydrolysis process. Thus, maltodextrin structure does not occur in nature.

Other possible excipients or carriers that may be added to the combination or mixture of the invention include arabic gum, dextrose, and salt.

The combinations or mixtures defined herein may be in the form of a powder.

The combinations or mixtures defined herein may also be provided in the form of a nutraceutical, pharmaceutical or food composition, comprising the combinations or mixtures defined herein.

Thus, the present invention provides a nutraceutical, pharmaceutical or food composition, comprising the combinations or mixtures defined herein.

Such nutraceutical, pharmaceutical or food compositions may be intended for use in or on humans.

As used herein the term “nutraceutical composition” is understood to mean that the composition is a pharmaceutical alternative, having a beneficial or protective physiological effect, for example, against chronic diseases.

The pharmaceutical, nutraceutical or food compositions may comprise a combination or mixture as defined herein in a therapeutically effective amount. As used herein, the term “effective amount” is synonymous with “therapeutically effective amount”, “effective dose”, or “therapeutically effective dose” and when used in reference to reducing the formation of atherosclerotic plaques and/or reducing LDL and/or increasing HDL, and/or decreasing isoprostanes, refers to the minimum dose of the combination or mixture defined herein necessary to achieve the desired therapeutic effect and includes a dose sufficient to reduce a symptom associated with the formation of atherosclerotic plaques and/or high LDL and/or low HDL, and/or high isoprostanes. Effectiveness in reducing the formation of atherosclerotic plaques and/or reducing LDL and/or increasing HDL, and/or decrease isoprostanes can be determined by observing an improvement in an individual based upon one or more clinical symptoms, and/or physiological indicators associated with the condition.

The compositions (e.g. a pharmaceutical, cosmetic, nutraceutical or food composition) of the invention advantageously comprise the extracts defined above in sufficient amounts to provide TCGAs in an amount of from about 20% to about 50% by weight of the composition, such as from about 30% to about 40% by weight of the composition; carnosic acid in an amount of from about 1% to about 20% by weight of the composition, such as from about 2% to about 10% by weight of the composition; and carnosol in an amount of from about 0.1% to about 5%, such as from about 0.2% to about 1% by weight of the composition.

The compositions according to the invention may contain at least one other active ingredient, in combination with the combination or mixture of the invention.

The compositions according to the invention may further comprise a physiologically acceptable excipient, adapted in particular according to the intended form and the desired route of administration of the composition.

As used herein, references to pharmaceutically acceptable excipients may refer to pharmaceutically acceptable adjuvants, diluents and/or carriers as known to those skilled in the art.

The physiologically acceptable excipient may be a food acceptable excipient.

Pharmaceutical/nutraceutical/food acceptable ingredients/excipients include those known in the art (including those also referred to herein as pharmaceutically acceptable excipients) and can be natural or non-natural, i.e. their structure may occur in nature or not. In certain instances, they can originate from natural compounds and be later modified so that it is distinct from the natural product from which it originated (e.g. maltodextrin).

Suitable carriers include, but are not limited to, inert solid diluents or fillers, sterile aqueous solutions and various organic solvents. Examples of solid carriers are lactose, terra alba, sucrose, cyclodextrin, maltodextrin, dextrin, talc, gelatine, agar, pectin, acacia, magnesium stearate, magnesium hydroxide; stearic acid, arabic gum, modified starch and lower alkyl ethers of cellulose, saccharose, silicon dioxide. Examples of liquid carriers are syrup, vegetables oils, phospholipids, fatty acids, fatty acid amines, polyoxyethylene and water. Moreover, the carrier or diluent may include any sustained release material known in the art, such as glyceryl monostearate or glyceryl distearate, alone or mixed with a wax.

Examples of other physiologically acceptable carriers that may be used in the compositions of the present invention include buffers such as phosphate, citrate, and other organic acids; antioxidants including ascorbic acid; low molecule weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN (for example, polysorbate based emulsifiers, such as polysorbate 20, 40, 60 or 80), polyethylene glycol (PEG), and PLURONIC (for example, block copolymers based on ethylene oxide and propylene oxide).

The skilled person will understand that compositions of the invention (e.g. in the form of compositions, such as pharmaceutical, nutraceutical or food compositions), the combination or the mixture of the present invention may be administered to a patient or subject (e.g. a human or animal patient or subject) by any suitable route, such as by the enteral, topical, oral, rectal, nasal, pulmonary, buccal, sublingual, transdermal, intracisternal, intraperitoneal, and parenteral (including subcutaneous, intramuscular, intrathecal, intravenous and intradermal) route.

For an enteral route, the compositions (more particularly the nutraceutical, food and/or pharmaceutical composition), combination or mixture may be in the form of tablets, gelatin capsules, dragées, syrups, suspensions, solutions, powders, granules, emulsions, microspheres or nanospheres, or lipid or polymeric vesicles permitting controlled release.

For a parenteral route, the compositions, combination or mixture may be in the form of solutions or suspensions, for perfusion or for injection.

The compositions, combination or mixture of the invention, used according to the invention may be administered in a daily dose of approximately from about 200 mg/day to about 800 mg/day of the combination or mixture, such as from about 250 mg/day to about 600 mg/day.

For example, the compositions, combination or mixture of the invention, used according to the invention may be administered in an amount of from about 2 mg/kg of body weight to about 20 mg/kg of body weight, such as from about 4 mg/kg to about 10 mg/kg.

The dosages indicated above would typically provide green coffee bean extract in an amount of from about 100 mg/day to about 500 mg/day, such as from about 200 mg/day to about 400 mg/day; rosemary extract in an amount from about 50 mg/day to about 200 mg/day, such as from about 75 mg/day to about 150 mg/day; chlorogenic acid in an amount of from about 68 mg/day to about 250 mg/day, such as from about 95 mg/day to about 195 mg/day, such as about 97 mg/day or about 193 mg/day; and carnosol and carnosic acid in an amount of from about 10 mg/day to about 43 mg/day, such as from about 15 mg/day to about 35 mg/day, such as about 16.5 mg/day to about 33 mg/day.

The dosages indicated above would typically provide green coffee bean extract in an amount of from about 2 mg/kg to about 10 mg/kg, such as from about 3 mg/kg to about 8 mg/kg; rosemary extract in an amount from about 0.5 mg/kg to about 5 mg/kg, such as from about 1 mg/kg to about 3 mg/kg; chlorogenic acid in an amount of from about 1 mg/kg to about 5 mg/kg, such as from about 1.5 mg/kg to about 3.5 mg/kg, such as about 1.6 mg/kg or about 3.22 mg/kg; and carnosol and carnosic acid in an amount of from about 0.1 mg/kg to about 2 mg/kg, such as from about 0.2 mg/kg to about 1 mg/kg, such as about 0.28 or about 0.55 mg/kg.

The compositions, combination or mixture of the invention may be formulated as a single composition comprising all of the compounds defined above or may be formulated as compositions comprising a single compound as defined above and the compositions administrated together.

Pharmaceutical, nutraceutical or food compositions of the invention may consist of or consist essentially of the combination or mixture of the invention.

For the avoidance of doubt, in this specification when we use the term “comprising” or “comprises” we mean that the extract or composition being described must contain the listed ingredient(s) but may optionally contain additional ingredients. When we use the term “consisting essentially of” or “consists essentially of” we mean that the extract or composition being described must contain the listed ingredient(s) and may also contain small (for example up to 5% by weight, or up to 1% or 0.1% by weight) of other ingredients provided that any additional ingredients do not affect the essential properties of the extract or composition. When we use the term “consisting of” or “consists of” we mean that the extract or composition being described must contain the listed ingredient(s) only.

As indicated previously in the application, the combination or mixture defined herein has been found to reduce the formation or atheroscleosic plaques, and/or decrease LDL and/or increase HDL and/or decrease isoprostanes.

The present invention provides a combination for use in reducing and/or preventing the formation of atherosclerotic plaques and/or reducing LDL levels and/or increasing HDL levels, and/or decrease isoprostanes (such as 8-iso-PGF2 α) levels

The present invention also provides a mixture for use in reducing and/or preventing the formation of atherosclerotic plaques and/or reducing LDL levels and/or increasing HDL levels, and/or decrease isoprostanes (such as 8-iso-PGF2 α) levels.

The present invention further provides a combination of the invention, a mixture of the invention or a composition according to the invention for use in reducing and/or preventing the formation of atherosclerotic plaques and/or reducing LDL levels and/or increasing HDL levels, and/or decrease isoprostanes (such as 8-iso-PGF2 α) levels.

Also provided is a method of reducing and/or preventing the formation of atherosclerotic plaques and/or reducing LDL levels and/or increasing HDL levels, and/or decrease isoprostanes (such as 8-iso-PGF2 α) levels, wherein the method comprises administering a mixture of the invention or a composition according to the invention to a patient in need thereof.

Use of a combination of the invention for the manufacture of a medicament for reducing and/or preventing the formation of atherosclerotic plaques and/or reducing LDL levels and/or increasing HDL levels, and/or decrease isoprostanes (such as 8-iso-PGF2 α) levels.

Further, the present invention provides the use of a composition or a mixture according to the invention in the manufacture of a medicament for use in reducing and/or preventing the formation of atherosclerotic plaques and/or reducing LDL levels and/or increasing HDL levels, and/or decrease isoprostanes (such as 8-iso-PGF2 α) levels.

Also provided is a method of reducing and/or preventing the formation of atherosclerotic plaques and/or reducing LDL levels and/or increasing HDL levels, and/or decrease isoprostanes (such as 8-iso-PGF2 α) levels, wherein the method comprises administering a composition comprising the combination or mixture of the invention to a patient in need thereof.

As mentioned before, atherosclerotic vasculopathy is a multifactorial process causing vessels damage and ardiovascular diseases and oxidized low-density lipoprotein (LDL) and 8-isoprostaglandin F2α belong to the most relevant risk factors for atherosclerosis plaques formation. The inventors of the present invention have demonstrated that the combination of the invention comprising an extract obtained or obtainable from a plant of the Coffea genus and a plant of the Lamiaceae family has a synergistic effect on HDL cholesterol (increase), a synergistic effect in the levels of oxidised LDL (decrease) and isoprostanes (decrease).

Thus, the present invention also provides a combination of the invention, a mixture of the invention or a composition according to the invention for use in preventing, alleviating and/or treating cardiovascular diseases and/or cerebrovascular diseases.

The present invention provides a combination of the invention, a mixture of the invention or a composition according to the invention for use in supporting cardiovascular and/or cerebrovascular health.

The present invention provides a method for preventing, alleviating and/or treating cardiovascular diseases and/or cerebrovascular diseases, wherein the method comprises administering a combination according to the invention, a mixture according to the invention or a composition according to the invention.

The present invention provides a method for supporting cardiovascular and/or cerebrovascular health, wherein the method comprises administering a combination according to the invention, a mixture according to the invention or a composition according to the invention.

The present invention provides the use of a combination of the invention, a mixture of the invention or a composition according to the invention in the manufacture of a medicament for supporting cardiovascular and/or cerebrovascular health.

The present invention provides the use of a combination of the invention, a mixture of the invention or a composition according to the invention in the manufacture of a medicament for preventing, alleviating and/or treating cardiovascular diseases and/or cerebrovascular diseases.

Cardiovascular diseases or complications include without limitation, coronary artery disease, coronary heart disease, hypertension, atherosclerosis, in particular iliac or femoral atherosclerosis, angina pectoris, thrombosis, heart failure, stroke, vascular aneurysm, vascular calcification, acute coronary syndromes such as myocardial infarction, vascular stenosis and infarction, and vascular dementia. Preferably, the cardiovascular disease according to the invention is selected from the group consisting of coronary artery disease, hypertension, atherosclerosis, vascular aneurysm, vascular calcification, vascular dementia and heart failure. More preferably, the cardiovascular disease according to the invention is atherosclerosis. Most preferably, the cardiovascular disease according to the invention is atherosclerotic carotid plaques.

Cerebrovascular diseases or complications (also named neurovascular diseases or complications) include, without limitation, brain aneurysms, arteriovenous malformations (AVMs), carotid artery disease, intracranial atherosclerotic disease, stroke and Transient Ischemic Attack (TIA).

In the uses and methods described herein, the composition, combination or mixture is administered in an amount sufficient to provide from about 50 mg/kg to about 120 mg/kg of the combination or mixture and/or from about 200 mg per day to about mg per day to about 600 mg per day of the combination or mixture.

In the uses and methods described herein, the composition, combination or mixture is administered in an amount sufficient to provide chlorogenic acid in an amount of from about 1 mg/kg to about 5 mg/kg, such as from about 1.5 mg/kg to about 3.5 mg/kg, such as about 1.6 mg/kg or about 3.22 mg/kg; and carnosol and carnosic acid in an amount of from about 0.1 mg/kg to about 2 mg/kg, such as from about 0.2 mg/kg to about 1 mg/kg, such as about 0.28 or about 0.55 mg/kg.

In the uses and methods described herein, the composition, combination or mixture is administered in an amount sufficient to provide chlorogenic acid in an amount of from about 68 mg/day to about 250 mg/day, such as from about 95 mg/day to about 195 mg/day, such as about 97 mg/day or about 193 mg/day; and carnosol and carnosic acid in an amount of from about 10 mg/day to about 43 mg/day, such as from about 15 mg/day to about 35 mg/day, such as about 16.5 mg/day to about 33 mg/day.

In one embodiment of the uses and methods described herein the subject is a mammal such as dogs, cats, cows, pigs, etc. In a preferred embodiment the subject is a male or a female human.

As already mentioned before, effectiveness in reducing the formation of atherosclerotic plaques and/or reducing LDL and/or increasing HDL, and/or decrease isoprostanes can be determined by observing an improvement in an individual based upon one or more clinical symptoms, and/or physiological indicators associated with the condition.

Thus, reducing and/or preventing the formation of atherosclerotic plaques in the present invention are understood as a statistically significant change in one or more indicia of atherosclerotic plaques. These indicia may include the reduction of the number of plaques, reduction in the size of plaques, halted development in the number of plaques, halted development in the size of plaques, between others well known in the art.

Thus in one embodiment of the methods and uses described herein, the administering of the composition, combination or mixture results in a statistically significant change in one or more indicia of atherosclerotic plaques. In another embodiment, the one or more indicia comprise one or more of reduction of the number of plaques, reduction in the size of plaques, halted development in the number of plaques, halted development in the size of plaques.

In another embodiment, the one or more indicia comprises enhanced proliferation of Treg (CD4+CD25+FoxP3+ and CD8+CD25+FoxP3+).

In another embodiment, the one or more indicia comprises production of one or more anti-inflammatory cytokines such as IL-10, IL-4 or TOPs.

Reducing and/or preventing the formation of atherosclerotic plaques in the present invention are understood as a statistically significant change in one or more of the above mentioned indicia.

Reducing or lowering the LDL levels is understood as reducing the blood levels of LDL from levels that are considered as high or risk levels compared to control levels. For example, LDL cholesterol control levels can be less than 100 mg/dL. Levels of 100 to 129 mg/dL may be acceptable for people with no health issues but may be of more concern for those with heart disease or heart disease risk factors. A reading of 130 to 159 mg/dL can be considered as borderline high and 160 to 189 mg/dL can be considered high. However the control and risk levels may depend on age and sex of the subject.

Increasing HDL levels is understood in the present application as the increase of the blood levels of HDL to a desirable level. For example 60 mg/dL (1.6 mmol/L) or above of HDL may be considered as desirable levels that are linked by the medical practitioners to healthy levels. The desirable level may depend on the sex and age of the subject.

Isoprostanes are well recognized markers of oxidative stress and thus “decreasing or lowering the levels of isoprostanes (such as 8-iso-PGF2 α)” is understood as to reach desirable levels (control levels or baseline levels). The methods for measuring those compounds in the blood are well known in the art.

The lowering of the parameter (i.e. LDL) can be address for example by administering to members of the subject group the composition of the invention, wherein upon administering the composition to members of the subject group daily for a certain period (for example about 12 weeks) the subject group exhibits: at least 10%, at least 15%, at least 20%, at least 25%, at least 30%, at least 35%, at least 40%, at least 45%, at least 50%, at least 55%, at least 60%, at least 65%, at least 70%, at least 75% lower mean of the parameter (i.e LDL) by comparison with a control subject group maintained on placebo therapy without treatment for said certain period (such as 12 weeks).

In one embodiment the lowering of the parameter (i.e LDL or isoprostanes level) is of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) as compared to baseline or placebo control.

In one embodiment the increase of the parameter (i.e HDL level) is of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) as compared to baseline or placebo control.

As used herein, the term “treatment” or grammatical equivalents encompasses the improvement and/or reversal of the symptoms of disease (e.g., heart disease). A composition which causes an improvement in any parameter associated with disease when used in the screening methods of the instant invention may thereby be identified as a therapeutic composition. The term “treatment” refers to both therapeutic treatment and prophylactic or preventative measures. For example, those who may benefit from treatment with compositions, uses and methods of the present invention include those already with a disease and/or disorder (e.g., elevated cholesterol levels) as well as those in which a disease and/or disorder is to be prevented (e.g., using a prophylactic treatment of the present invention).

The term “prevention” in relation to a given disease or disorder means: preventing the onset of disease development if none had occurred, preventing the disease or disorder from occurring in a subject that may be at risk or predisposed to the disorder or disease but has not yet been diagnosed as having the disorder or disease, and/or preventing further disease/disorder development if already present.

As used herein, the term “at risk for disease” refers to a subject (e.g., a human) that is predisposed to experiencing a particular disease. This predisposition may be genetic (e.g., a particular genetic tendency to experience the disease, such as heritable disorders), or due to other factors (e.g., age, weight, environmental conditions, exposures to detrimental compounds present in the environment, etc.). Thus, it is not intended that the present invention be limited to any particular risk, nor is it intended that the present invention be limited to any particular disease.

A therapeutically effective amount of any embodiment of the present invention is determined using methods known to pharmacologists and clinicians having ordinary skill in the art. For example, an effective amount can be determined subjectively by administering increasing amounts of the compositions of the present invention until such time the patient being treated shows reduction in LDL cholesterol levels. Blood levels of the composition, cholesterol and lipid levels can be determined using routine biological and chemical assays and these blood levels can be matched to the route of administration. The blood level and route of administration giving the most desirable level of cholesterol reduction can then be used to establish an “effective amount” of the pharmaceutical composition for treatment.

The present invention also provides a method for producing a combination or mixture of the invention.

For the avoidance of doubt, preferences, options, particular features and the like indicated for a given aspect, feature or parameter of the invention should, unless the context indicates otherwise, be regarded as having been disclosed in combination with any and all other preferences, options particular features and the like as indicated for the same or other aspects, features and parameters of the invention.

The term “about” as used herein, e.g. when referring to a measurable value (such as an amount or weight of a particular component in the reaction mixture), refers to variations of ±20%, 10%, 5%, 1%, 0.5%, or, particularly, ±0.1% of the specified amount.

The invention will now be described by reference to the following, non-limiting, figures and examples.

### BRIEF DESCRIPTION OF THE FIGURES

FIG. 1: Typical software calculation of the a theromatic plaques (atherosclerotic plaque) area.

FIG. 2: Comparison of the “Lesions Area” (in % of the total aorta area) between the vehicle, green coffee extract and rosemary diets with the diets of the combination 1 or combination 2 of the invention.

FIG. 3: Cryosection of aorta from the mouse #64 showing atherosclerotic plaque (VEHICLE group).

FIG. 4: Comparison of the vehicle, green coffee extract and rosemary diets with a diet of the combination or mixture of the invention on the of mean values for the variable “Total Cholesterol” (in mg/dL).

FIG. 5: Comparison of the vehicle, green coffee extract and rosemary diets with a diet of the combination or mixture of the invention on the of mean values for the variable “HDL-Cholesterol” (in mg/dL).

FIG. 6: Comparison of the vehicle, green coffee extract and rosemary diets with a diet of the combination or mixture of the invention on the of mean values for the variable “Non HDL-Cholesterol” (in mg/dL).

FIG. 7: Comparison of the vehicle, green coffee extract and rosemary diets with a diet of the combination or mixture of the invention on the of mean values for the variable “Oxidized LDL-Cholesterol” (in mg/dL).

FIG. 8: Comparison of the vehicle, green coffee extract and rosemary diets with a diet of the combination or mixture of the invention on the of mean values for the variable “PGF2” (in pg/mL).

### EXAMPLES

Material and Methods

Animal Study

Five groups of mice have been fed with the same standardized diet (55% carbohydrates, 13% lipids and 32% proteins, water ad libidum) and were also administered different products via daily intragastric gavage for 16 weeks (experiments performed in Biomeostasis, Marseille, France):
Group 1: Suspension of 0.5% w/v carboxymethylcellulose (CMC) VEHICLE
Group 2: green coffee extract, 83 mg/kg body weight, purchased by Naturex, Avignon, France and added to the vehicle (0.5% CMC suspension). Experiment name SVETOL.
Group 3: rosemary extract, 31 mg/kg body weight, purchased by Naturex, Avignon, France and added to the vehicle (0.5% CMC suspension). Experiment name ROSEMARY.
Group 4: mixture of green coffee and rosemary extracts, 114 mg/kg body weight, purchased by Naturex, Avignon, France and added to the vehicle (0.5% CMC suspension). Experiment name COMBINATION DOSE 1 (comprising 27% rosemary ethanol extract and 73% green coffee bean hydro-ethanolic extract). Group 5: mixture of green coffee and rosemary extracts, 57 mg/kg body weight, purchased by Naturex, Avignon, France and added to the vehicle (0.5% CMC suspension). Experiment name COMBINATION DOSE 2 (comprising 27% rosemary ethanol extract and 73% green coffee bean hydro-ethanolic extract

SVETOL is a green coffee extract (Coffea canephora Pierre ex A. Froehner syn. Coffea robusta L. Linden) extracted using an water (70%) and ethanol (30%) and has total chologenica acid content of 45-50%, 5-caffeoylquinic acid (5-CQA) content of 10-17% and a 5-caffeoylquinic acid/total chologenic acid ratio of 0.2-0.3 The caffeine content is less than 2%.

For example, one sample of svetol has the following concentration of CGAs: about 8% of 3-CQA, about 13.2% of 5-CGA (5-CQA), about 2% of 3-FQA, about 9.4% of 4-CQA, about 3.3% of 4-FQA, about 2.3% of 5FQA, about 2.44 of 3.4-di-CQA, about 1.-6% of 3.5-di-CQA, about 3.8% e-2 of 4.5-di-QA, about 3.1% of 3.4-CFQA, about 0.4% of 3.5-CFQA, about 0.9% of 4.5-CFQA, about 0.5% of C-try and/or about 8.2 e-2% of p-coumatryl-tryp ROSEMARY is a Rosmarinus officinalis alcoholic extract that has a carnosic acid and carnosol content of 21.5-25% and a carnosic acid content of 20-22%.

Histology and Lesion Analysis

At the end of the treatment periods, mice were anesthetized (150 mg/kg ketamine, 10 mg/kg xylazine) and were exsanguinated by left ventricle puncture for lipid profile analysis and atherosclerotic plaque formation in the aorta. For “en face” analysis, the aorta were dissected out, opened longitudinally from heart to the iliac arteries, and stained with Sudan IV to determine lesion area according to Collins et al. (2001) Arterioscler. Thromb. Vasc. Biol. 21, 365-371. Images were scanned by use of a NanoZoomer slide scan (Hamamatsu, Japan) and analysed by a single technician (who was blinded to the study protocol) using IMAGE J analysis software. The extent of lesion formation was expressed as the percentage of the total aortic surface area covered by lesions.

Triglycerides, Total Cholesterol, HDL and LDL Cholesterol and Oxidized LDL

Blood samples were drawn on EDTA for determination of triglycerides, total cholesterol, HDL and LDL cholesterol and oxidized LDL (OxLDL). Kits for measurement of total cholesterol (CHOL2), HDL-cholesterol (HDLC3), LDL-cholesterol (LDLC3) and triglycerides (TRIGLY) were purchased from Roche. All analyses were performed on Cobas device.

Oxidized Low Density Lipoproteins (OxLDL) were determined by the ELISA assay purchased by Kamiya Biomedical Company, Seattle USA. The microtiter plate provided in this kit has been pre-coated with an antibody specific to OxLDL. Calibrators or samples are then added to the appropriate microtiter plate wells with a biotin-conjugated antibody preparation specific for OxLDL. Next, Avidin conjugated to Horseradish Peroxidase (HRP) is added to each microplate well and incubated. Then a TMB substrate solution is added to each well. Only those wells that contain OxLDL, biotin-conjugated antibody and enzyme-conjugated Avidin will exhibit a change in color. The enzyme-substrate reaction is terminated by the addition of a sulfuric acid solution and the color change is measured spectrophotometrically at a wavelength of 450 nm±10 nm. The concentration of OxLDL in the samples is then determined by comparing the optical density (O.D.) of the samples to the calibration curve.

The Detection Range was 31.2-2,000 pg/mL. The minimum detectable dose of mouse OxLDL is typically below 13.9 pg/mL. The sensitivity of this assay, or Lower Limit of Detection (LLOD) was defined as the lowest protein concentration that could be differentiated from zero. It was determined the mean O.D. Value of 20 replicates of the zero calibrator plus three standard deviations. This assay has high sensitivity and excellent specificity for detection of mouse OxLDL. No significant cross-reactivity or interference between mouse OxLDL and analogues was observed. Note: Limited by current skills and knowledge, it is impossible for us to complete the cross-reactivity detection between mouse OxLDL and all the analogues, therefore, cross reaction may still exist.

Polyphenol Determination

Test Items

Carnosic acid, including the certificate of analysis, was supplied by Chromadex Inc. (Irvine, USA). The supplier is responsible for the identity and quality of this test item.
Molecular Formula: C₂₀H₂₈O₄, Molecular weight: 332.43 g/mol. Lot Number: 00003198-081. CAS Number: 3650-09-7. Assay: 97.0% (HPLC)

Carnosol, including the certificate of analysis, was supplied by Chromadex Inc. (Irvine, USA). The supplier is responsible for the identity and quality of this test item.
Molecular Formula: C₂₀H₂₆O₄. Molecular weight: 330.42 g/mol. Lot Number: 00003199-080. CAS Number: 5957-80-2. Assay: 98.7% (HPLC)

Chlorogenic acid, including the certificate of analysis, was supplied by Extrasynthese (Lyon, FRANCE). The supplier is responsible for the identity and quality of this test item.
Molecular Formula: C₁₆H₁₈O₉. Molecular weight: 354.31 g/mol. Lot Number: Batch 08. CAS Number: 327-97-9. Assay: 100% (HPLC)

Cafeic acid, including the certificate of analysis, was supplied by Extrasynthese (Lyon, FRANCE). The supplier is responsible for the identity and quality of this test item.
Molecular Formula: C₉H₈O₄. Molecular weight: 180.16 g/mol. Lot Number: Batch 1. CAS Number: 331-39-5. Assay: 100% (HPLC)

On their reception, the test items were checked and registered along with all relevant details and remarks related to the condition of the product. They were stored according to the instructions of the supplier.

Internal Standard

PGF₂-D9, including the certificate of analysis, was supplied by Cayman Chemicals Company (Ann Arbor, USA). The supplier is responsible for the identity and quality of this compound, which was used as internal standard (IS).
Molecular Formula: C₂₀H₂₅D₉O₅. Molecular weight: 363.50 g/mol. Lot Number: 200090. Assay: 100.0% (HPLC)

On its reception, the internal standard was checked and registered along with all relevant details and remarks related to the condition of the product. It was stored according to the instructions of the supplier.

Apparatus and Equipment

All equipment were maintained and calibrated according to ATC procedures, and calibration period were controlled before any use.

Reagents

The solvents and reagents required and used were of analytical grade or equivalent. Their suppliers can be found in the tables below:

| Reagent | Supplier |
|---|---|
| Acetonitrile, LC/MS | Biosolve |
| Dimethyl sulfoxide, ACS | Merck |
| Sodium acetate, for analysis | Merck |
| Acetic acid, 100% | VWR |
| Formic acid, ULC/MS | Biosolve |
| Ascorbic acid, for analysis | Fagron |
| β-glucoronidase/arylsulfatase | Roche |
| Milli-Q water | — |

Liquid Chromatography
Analytical column: Waters Acquity UPLC HSS C₁₈ SB, L=100 mm & ID=2.1 mm, particle size=1.8 μm preceded by a precolumn filter.
Mobile phases: A: Water+0.1% formic acid.
   B: Acetonitrile+0.1% formic acid
Injection volume: maximum 0.1 μL.
Flow rate: 0.65 mL/min.
Gradient: linear gradient followed by an equilibrium period under the initial conditions prior to injection of the next sample

Mass Spectrometry Parameters

The mass spectrometer was a Waters Xevo TQ-S mass spectrometer equipped with an electrospray ionisation source (ESI) operating in negative ion mode and interfaced with a Waters Acquity UPLC I-Class inlet system. Data acquisition was achieved using MassLynx Version 4.1 software and QuanLynx version 4.1 software.

The acquisition was performed in the multiple reaction monitoring (MRM) mode. One specific transition was monitored for the quantification of each compound of interest. The selection of the transitions was made in order to reach the highest detectability and selectivity.

The main parameters for mass spectrometry were as follows:
Capillary: 2.50 kV.
Source temperature: 150° C.
Desolvation temperature: 650° C.
Cone gas flow (L/h): 150
Desolvation gas flow (L/h): 1000
Transition (m/z), cone voltage (V) and collision energy (eV):

| Analyte | Transition | Cone voltage | Collision energy |
|---|---|---|---|
| Carnosic acid | 331 > 287 | 28 | 16 |
| Carnosol | 329 > 285 | 28 | 24 |
| Chlorogenic acid | 353 > 191 | 28 | 25 |
| Cafeic acid | 179 > 135 | 28 | 15 |
| PGF₂-D9 | 362 > 318 | 35 | 19 |

Standard Solutions

The detailed preparation procedure of the different standard solutions is described below.

Stock Solutions

Since the chlorogenic acid is not stable in solution, a stock solution of each analyte was prepared prior to start of each study day, by dissolution of each test item in DMSO in order to obtain a concentration of 1 mg/mL (ST1 to ST4).

Prior the start of the study, an internal standard's stock solution was prepared. PGF₂-D9 was diluted in DMSO in order to obtain a concentration of 1 mg/mL. This solution was kept below −20° C.

Working Solutions

Two series of working solutions were diluted for each study day. A first one to quantify chlorogenic acid and carnosic acid. And a second one for cafeic acid and carnosol.

Dilution of the stock solutions were made in order to reach the following concentration levels: 10.000, 4.000, 1000, 200, 20 and 10 ng/mL for calibration. Dilutions were made with DMSO as shown in the following table:

Working solutions

| Standard | | Dilution | Final | |
|---|---|---|---|---|
| Solution | Diluted Solution/Volume | Volume | Concentration | |
| CAL0 | 2 of the 4 stock solutions/ | 900 μL | 50 | μg/mL |
| | 50 μL | | | |
| CAL1 | CAL0/200 μL | 800 μL | 10.000 | ng/mL |
| CAL2 | CAL1/400 μL | 600 μL | 4000 | ng/mL |
| CAL3 | CAL2/250 μL | 750 μL | 1000 | ng/mL |
| CAL4 | CAL3/200 μL | 800 μL | 200 | ng/mL |
| CAL5 | CAL4/100 μL | 900 μL | 20 | ng/mL |
| CAL6 | CAL5/300 μL | 300 μL | 10 | ng/mL |

For all test items, the same internal standard was used. PGF₂-D9 at 1 mg/mL was diluted in DMSO in order to obtain a working solution at a concentration of 5.000 ng/mL.

All these working solutions were used to spike blank plasma matrices. They were renewed each study day.

Sample Preparation

First, acetate buffer is prepared by mixing 2.05 g of sodium acetate, 0.5 g of ascorbic acid and 250 mL of water. The pH is then adjusted to 5 by adding acetic acid. The buffer is kept in the refrigerator, protected from light, for a maximum of 1 week.

In a 1.5 mL Eppendorf tube, introduce 50 μL of plasma, 100 μL of the buffer and 10 μL of a ß-glucoronidase/arylsulfatase solution (diluted 8× in water). After mixing, the tube is placed at 37° C. for 2 hours in a thermostatic oven.

To stop the hydrolysis and extract the analytes of interest, 500 μL of acetonitrile is added. Internal standard is introduced (10 μL of the working solution) and 25 μL of the adequate working solution (if needed). After mixing, the tube is centrifuged (10 minutes, 25.000 g, 4° C.).

The supernatant is transferred in another tube in order to be placed in the centrifuge concentrator, until (almost) dryness.

250 μL of acetonitrile and 250 μL of water are added. The tube is vortexed, 200 μL are transferred in glass vials (with an insert) for injection.

Isoprostanes Determination

Test Item

PGF₂, including the certificate of analysis, was supplied by Cayman Chemicals Company (Ann Arbor, USA). The supplier is responsible for the identity and quality of this test item.
Molecular Formula: C₂₀H₃₄O₅
Molecular weight: 354.50 g/mol
Lot Number: 198589
Assay: 100.0% (HPLC)
Storage conditions: below −20° C.

On its reception, the test item was checked and registered along with all relevant details and remarks related to the condition of the product. It was stored according to the instructions of the supplier.

Internal Standard

PGF₂-D9, including the certificate of analysis, was supplied by Cayman Chemicals Company (Ann Arbor, USA). The supplier is responsible for the identity and quality of this compound, which was used as internal standard (IS).
Molecular Formula: C₂₀H₂₃D₉O₅
Molecular weight: 361.50 g/mol
Lot Number: 0422307
Assay: 95.8% (HPLC)
Storage conditions: Below −20° C.

On its reception, the internal standard was checked and registered along with all relevant details and remarks related to the condition of the product. It was stored according to the instructions of the supplier.

Reagents

The solvents and reagents required and used were of analytical grade or equivalent. Their suppliers can be found in the table below:

| Reagent | Supplier |
|---|---|
| Acetonitrile, LC/MS | Biosolve |
| Methanol, LC/MS | Biosolve |
| Isopropanol, LC/MS | Biosolve |
| Ethyl acetate, LC/MS | VWR |
| Dimethyl sulfoxide, ACS | Merck |
| Potassium hydroxide, for analysis | Merck |
| Formic acid, ULC/MS | Biosolve |
| Milli-Q water | — |

Liquid Chromatography
Analytical column: Phenomenex Luna Omega Polar C₁₈, L=100 mm & ID=2.1 mm, particle size=1.6 μm preceded by a Phenomenex SecurityGuard Ultra C₁₈ for 2.1 mm ID column.
Mobile phases: A: Water+0.05% acetic acid.
   B: Acetonitrile+0.05% acetic acid
Injection volume: maximum 5 μL.
Flow rate: 0.40 mL/min.
Gradient: linear gradient followed by an equilibrium period under the initial conditions prior to injection of the next sample.

Mass Spectrometry Parameters

The mass spectrometer was a Waters Xevo TQ-S mass spectrometer equipped with an electrospray ionisation source (ESI) operating in negative ion mode and interfaced with a Waters Acquity UPLC I-Class inlet system. Data acquisition was achieved using MassLynx Version 4.1 software and QuanLynx version 4.1 software.

The acquisition was performed in the multiple reaction monitoring (MRM) mode. One specific transition was monitored for the quantification of each compound of interest. The selection of the transitions was made in order to reach the highest detectability and selectivity.

The main parameters for mass spectrometry were as follows:
Capillary: 2.27 kV.
Source temperature: 150° C.
Desolvation temperature: 650° C.
Cone gas flow (L/h): 150
Desolvation gas flow (L/h): 1000
Transition (m/z), cone voltage (V) and collision energy (eV):

| Analyte | Transition | Cone voltage | Collision energy |
|---|---|---|---|
| PGF₂ | 353 > 193 | 35 | 27 |
| PGF₂-D9 | 362 > 318 | 35 | 19 |

Standard Solutions

The detailed preparation procedure of the different standard solutions is described below.

Stock Solutions

Prior the start of the study, a PGF₂ stock solution was prepared. PGF₂ was diluted in DMSO in order to obtain a concentration of 500 ng/mL. This solution was kept below −20° C.

The exact same procedure was followed for the internal standard. PGF₂-D9 was diluted in DMSO in order to obtain a concentration of 1 mg/mL. This solution was kept below −20° C.

Working Solutions

Dilution of the stock solutions were made in order to reach the following concentration levels: 10.000, 2.500, 1250, 500, 250, 125 and 50 pg/mL for calibration. Dilutions were made with DMSO as shown in the following table:

Working solutions

| Standard | Diluted | Dilution | Final | |
|---|---|---|---|---|
| Solution | Solution/Volume | Volume | Concentration | |
| CAL1 | Stock solution/100 μL | 4900 μL | 10.000 | pg/mL |
| CAL2 | CAL1/2000 μL | 6000 μL | 2500 | pg/mL |
| CAL3 | CAL2/3000 μL | 3000 μL | 1250 | pg/mL |
| CAL4 | CAL3/2000 μL | 3000 μL | 500 | pg/mL |
| CAL5 | CAL4/2000 μL | 2000 μL | 250 | pg/mL |
| CAL6 | CAL5/2000 μL | 2000 μL | 125 | pg/mL |
| CAL7 | CAL6/1000 μL | 1500 μL | 50 | pg/mL |

The internal standard's stock solution was diluted in DMSO in order to obtain a working solution at a concentration of 5.000 pg/mL.

All these working solutions were used to spike blank plasma matrices. They were renewed each study day.

Sample Preparation

In a 1.5 mL Eppendorf tube, introduce 100 μL of plasma, 100 μL of KOH (1M in water), 20 μL of internal standard and 20 μL of the adequate working solution (if needed). After mixing, the tube is placed at 40° C. for 35 minutes in a thermostatic oven.

To stop hydrolysis, 100 μL of formic acid (11%) and 100 μL water (formic acid 1%) are added. The resulting mixture is then treated according to a sample preparation method developed by ATC. When dried, the final extracts are solubilized with 100 μL of a mixture of isopropanol, methanol, acetonitrile and water (1/1/1/1; v/v/v/v) for analysis.

Statistical Analysis

Differences between groups were evaluated by independent t tests (p<0.05 deemed significant) using Rcmdr software.

Results

En Face Analysis of Aorta

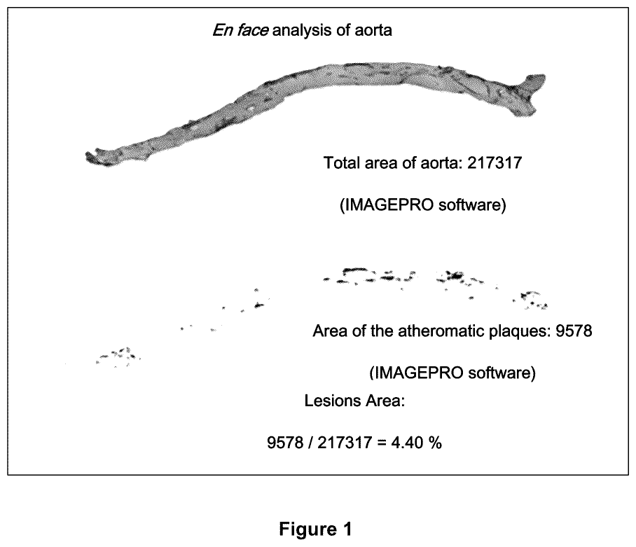

FIG. 1 shows a typical en face preparation of blood vessels image of mice apoE-submitted to the vehicle diet during 16 weeks (experiment performed in Liege). Using the IMAGEPRO software, the whole area of the aorta was first calculated. After treatment image, the total area of atherosclerotic lesions was calculated. The percentage of lesions area was calculated as the area ratio between the atherosclerotic plaques and the whole aorta.

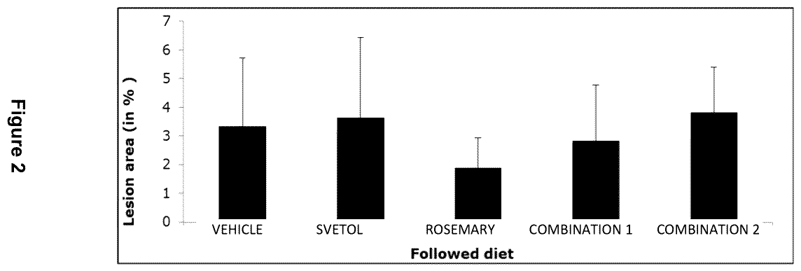

Table 1 and FIG. 2 summarize all data linked to the evaluation of the lesions area in the aorta.

TABLE 1
Results of the measurement of the lesions
areas (in % of the total aorta area).

| | | | | COMBI- | COMBI- |
|---|---|---|---|---|---|
| | | | | NATION | NATION |
| | VEHICLE | SVETOL | ROSEMARY | DOSE 1 | DOSE 2 |
| | 8.06 | 2.62 | 2.00 | 0.14 | 6.43 |
| | 1.27 | 3.11 | 0.77 | 1.50 | 1.42 |
| | 2.98 | 5.30 | 1.68 | 3.19 | 2.36 |
| | 7.24 | 0.73 | 0.70 | 1.12 | 4.75 |
| | 0.98 | 8.97 | 1.07 | 3.66 | 2.71 |
| | 3.44 | 7.63 | 1.74 | 3.84 | 2.93 |
| | 1.46 | 0.98 | 3.75 | 6.85 | 1.49 |
| | 4.10 | 3.43 | 1.46 | 4.21 | 3.52 |
| | 2.80 | 1.62 | 2.33 | 2.33 | 3.82 |
| | 1.13 | 1.99 | 3.47 | 1.47 | No Sample |
| Mean | 3.35 | 3.64 | 1.90 | 2.83 | 3.82 |
| SD | 2.38 | 2.80 | 1.04 | 1.94 | 1.59 |

A very large dispersion was observed with data of all groups. A tendency to a decrease of aorta lesions was evidenced with the ROSEMARY diet when compared to the VEHICLE group but without reaching a significant difference.

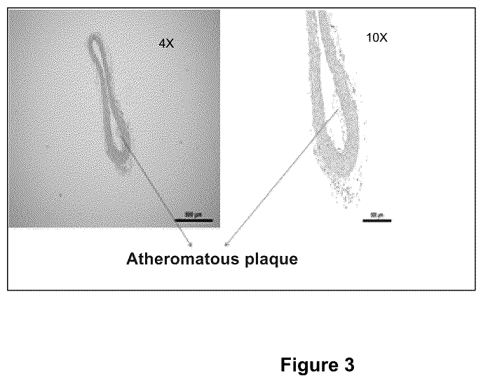

Due to the aorta fragmentation, the aortic root section analysis of atherosclerosis was not possible. For information, we show (in FIG. 3) for a mouse of the VEHICLE group a photography of a 4 μm-thick cryosections of aorta, beginning at the aortic root, collected for a distance of 400 μm. Sections were stained with Oil Red-O and hematoxylin.

Triglycerides, Total Cholesterol, HOL and LOL Cholesterol and Oxidized LOL

The individual results, mean values and standard deviations as expressed in mg/dL for the 5 diet groups are shown in Tables 2 to 6.

TABLE 2
Obtained results (in mg/dL) for VEHICLE diet.

| | | Total | HDL- | Non HDL- | HDL/Total |
|---|---|---|---|---|---|
| Mouse | Triglyc- | Choles- | Choles- | Choles- | Choles- |
| # | erides | terol | terol | terol | terol |
| 1 | 24 | 691 | 90 | 601 | 0.13 |
| 2 | 102 | 546 | 110 | 436 | 0.20 |
| 3 | 148 | 471 | 102 | 369 | 0.22 |
| 4 | 132 | 588 | 94 | 494 | 0.16 |
| 5 | 78 | 541 | 114 | 427 | 0.21 |
| 41 | 103 | 538 | 95 | 443 | 0.18 |
| 42 | 133 | No | 104 | No | No |
| | | Sample | | Sample | Sample |
| 43 | 95 | 569 | 105 | 464 | 0.18 |
| 44 | 89 | No | No | No | No |
| | | Sample | Sample | Sample | Sample |
| 45 | 87 | 500 | 102 | 398 | 0.20 |
| Mean | 99 | 556 | 102 | 454 | 0.19 |
| SD | 35 | 66 | 7.7 | 71 | 0.03 |

TABLE 3
Obtained results (in mg/dL) for SVETOL diet.

| | | Total | HDL- | Non-HDL | HDL/Total |
|---|---|---|---|---|---|
| Mouse | Triglyc- | Choles- | Choles- | Choles- | Choles- |
| # | erides | terol | terol | terol | terol |
| 6 | 128 | 511 | 108 | 403 | 0.21 |
| 7 | 99 | 568 | 360 | 208 | 0.63 |
| 8 | 61 | 457 | 108 | 349 | 0.24 |
| 9 | 58 | 428 | 96 | 332 | 0.22 |
| 10 | 58 | 455 | 104 | 351 | 0.23 |
| 31 | 72 | 503 | 116 | 387 | 0.23 |
| 32 | 196 | 803 | 226 | 577 | 0.28 |
| 33 | 90 | 500 | 320 | 180 | 0.64 |
| 34 | 75 | 471 | 103 | 368 | 0.22 |
| 35 | 107 | 567 | 104 | 463 | 0.18 |
| Mean | 94 | 526 | 165 | 362 | 0.31 |
| SD | 43 | 108 | 100 | 114 | 0.17 |

TABLE 4
Obtained results (in mg/dL) for ROSEMARY diet.

| | | Total | HDL- | Non HDL- | HDL/Total |
|---|---|---|---|---|---|
| Mouse | Triglyc- | Choles- | Choles- | Choles- | Choles- |
| # | erides | terol | terol | terol | terol |
| 11 | 178 | 632 | 110 | 532 | 0.17 |
| 12 | 115 | 562 | 115 | 447 | 0.20 |
| 13 | 74 | 411 | 101 | 310 | 0.25 |
| 14 | 78 | 397 | 97 | 300 | 0.24 |
| 15 | 106 | 529 | 100 | 429 | 0.19 |
| 46 | 93 | 511 | 107 | 404 | 0.21 |
| 47 | 80 | 469 | 115 | 354 | 0.25 |
| 48 | 83 | 499 | 340 | 159 | 0.68 |
| 49 | 156 | No | No | No | No |
| | | Sample | Sample | Sample | Sample |
| 50 | 149 | 471 | 93 | 378 | 0.20 |
| Mean | 111 | 498 | 131 | 368 | 0.27 |
| SD | 37 | 73 | 79 | 106 | 0.16 |

TABLE 5
Obtained results (in mg/dL) for COMBINATION DOSE 1 diet.

| | | Total | HDL- | Non HDL- | HDL/Total |
|---|---|---|---|---|---|
| Mouse | Triglyc- | Choles- | Choles- | Choles- | Choles- |
| # | erides | terol | terol | terol | terol |
| 16 | 60 | 400 | 106 | 294 | 0.26 |
| 17 | 83 | 424 | 93 | 331 | 0.22 |
| 18 | 76 | 545 | 113 | 432 | 0.21 |
| 19 | 88 | 556 | 109 | 447 | 0.2 |
| 20 | 60 | 405 | 84 | 321 | 0.21 |
| 26 | 55 | 436 | 102 | 334 | 0.23 |
| 28 | 104 | 612 | 360 | 252 | 0.59 |
| 29 | 126 | 477 | 113 | 364 | 0.24 |
| 30 | 100 | 688 | 380 | 308 | 0.55 |
| Mean | 84 | 505 | 162 | 343 | 0.30 |
| SD | 21 | 101 | 118 | 63 | 0.15 |

TABLE 6
Obtained results (in mg/dL) for COMBINATION DOSE 2 diet.

| | | Total | HDL- | Non HDL- | HDL/Total |
|---|---|---|---|---|---|
| Mouse | Triglyc- | Choles- | Choles- | Choles- | Choles- |
| # | erides | terol | terol | terol | terol |
| 21 | 200 | 718 | 115 | 603 | 0.16 |
| 22 | 167 | 570 | 118 | 452 | 0.21 |
| 23 | 69 | 397 | 109 | 288 | 0.27 |
| 24 | 110 | 616 | 360 | 256 | 0.58 |
| 25 | 113 | 614 | 102 | 512 | 0.17 |
| 36 | 139 | 475 | 90 | 385 | 0.19 |
| 37 | 154 | 662 | 360 | 302 | 0.54 |
| 38 | 126 | 467 | 226 | 241 | 0.48 |
| 39 | 164 | 707 | 420 | 287 | 0.59 |
| 40 | 110 | 564 | 116 | 448 | 0.21 |
| Mean | 135 | 579 | 202 | 377 | 0.34 |
| SD | 37 | 106 | 130 | 122 | 0.18 |

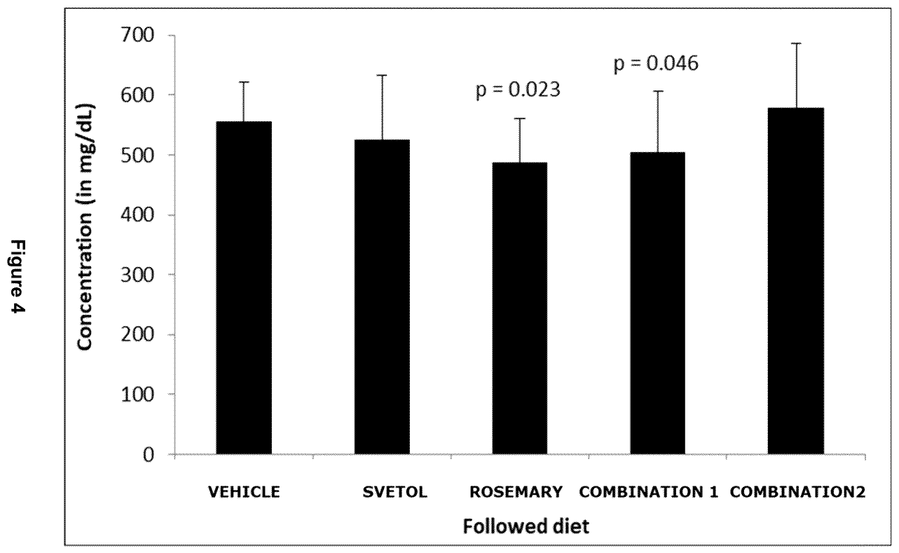

Table 7 and FIG. 4 show the comparison of mean values of the variable “Total Cholesterol” between all diet groups.

TABLE 7
Comparison of mean values for the variable
“Total Cholesterol” (in mg/dL).

| | | | | COMBI- | COMBI- |
|---|---|---|---|---|---|
| | | | | NATION | NATION |
| | VEHICLE | SVETOL | ROSEMARY | DOSE 1 | DOSE 2 |
| Mean | 556 | 526 | 488 | 505 | 579 |
| SD | 66 | 108 | 73 | 101 | 106 |

When compared to the VEHICLE group, a significant decrease of respectively 13% and 10% was observed with groups ROSEMARY (p=0.023) and COMBINATION DOSE 1 (p=0.046). No difference was observed with the two other groups.

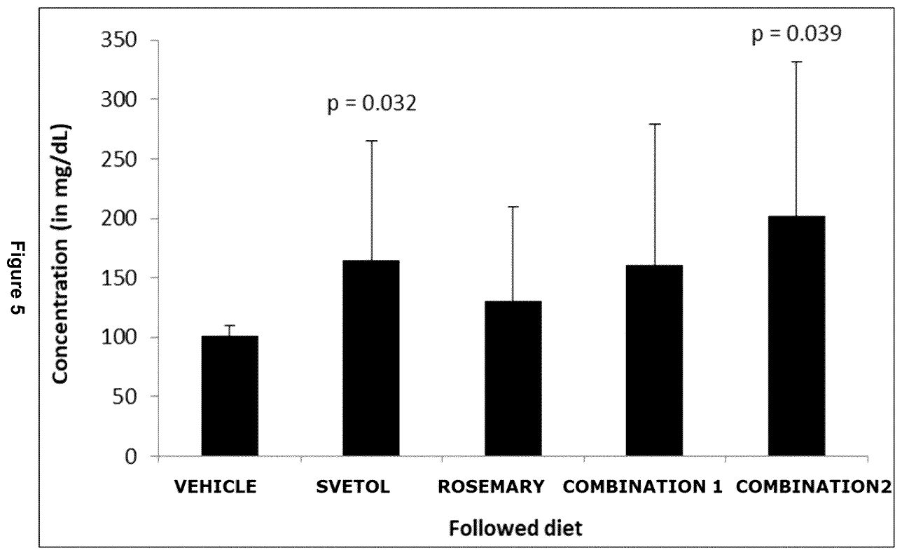

Table 8 and FIG. 5 show the comparison of mean values of the variable “HDL-Cholesterol” between all diet groups.

TABLE 8
Comparison of mean values for the variable
“HDL-Cholesterol” (in mg/dL).

| | | | | COMBI- | COMBI- |
|---|---|---|---|---|---|
| | | | | NATION | NATION |
| | VEHICLE | SVETOL | ROSEMARY | DOSE 1 | DOSE 2 |
| Mean | 102 | 165 | 131 | 161 | 202 |
| SD | 7.7 | 100 | 79 | 118 | 130 |

When compared to the VEHICLE group, the mean concentration of HDL-cholesterol was increased by 1.6 fold in groups SVETOL and COMBINATION DOSE 1 and, overall, by a factor 2 in the group COMBINATION DOSE 2. The increase was very moderate in the ROSEMARY group. Statistical analysis revealed that the increase observed in both groups SVETOL (p=0.032) and COMBINATION DOSE 2 (p=0.039) was statistically different when compared to VEHICLE group. This is a very surprising and positive effect since the lower dose of the combination show better results than all products alone used at a higher dosage, thus demonstrating synergy

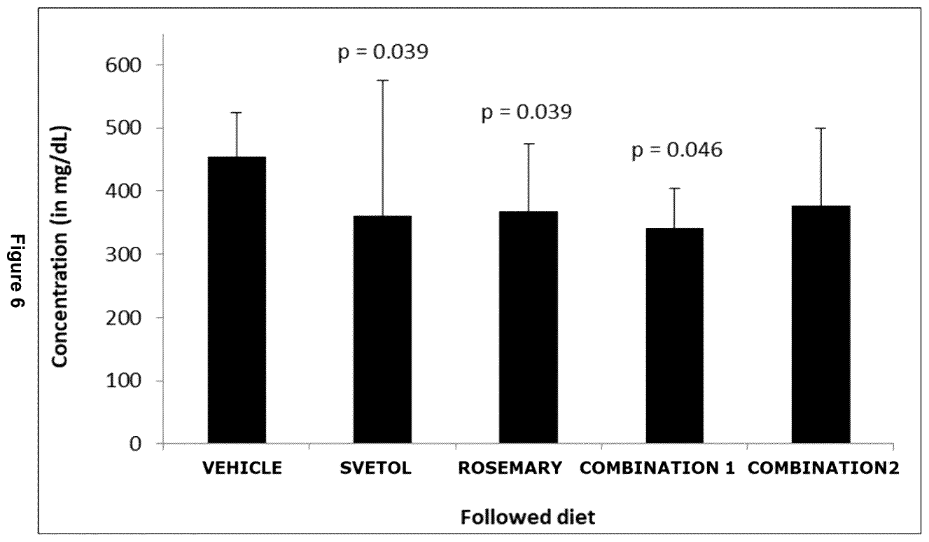

Table 9 and FIG. 6 show the comparison of mean values of the variable “Non HDL-Cholesterol” between all diet groups.

TABLE 9
Comparison of mean values for the variable “Non
HDL-Cholesterol” (in mg/dL).

| | | | | COMBI- | COMBI- |
|---|---|---|---|---|---|
| | | | | NATION | NATION |
| | VEHICLE | SVETOL | ROSEMARY | DOSE 1 | DOSE 2 |
| Mean | 454 | 362 | 368 | 343 | 377 |
| SD | 71 | 214 | 106 | 63 | 122 |

All intervention diets contributed to decrease the mean concentration of Non HDL-Cholesterol when compared to the VEHICLE group. The more important and significant effect was observed with SVETOL (21%) and COMBINATION DOSE 1 (25%).

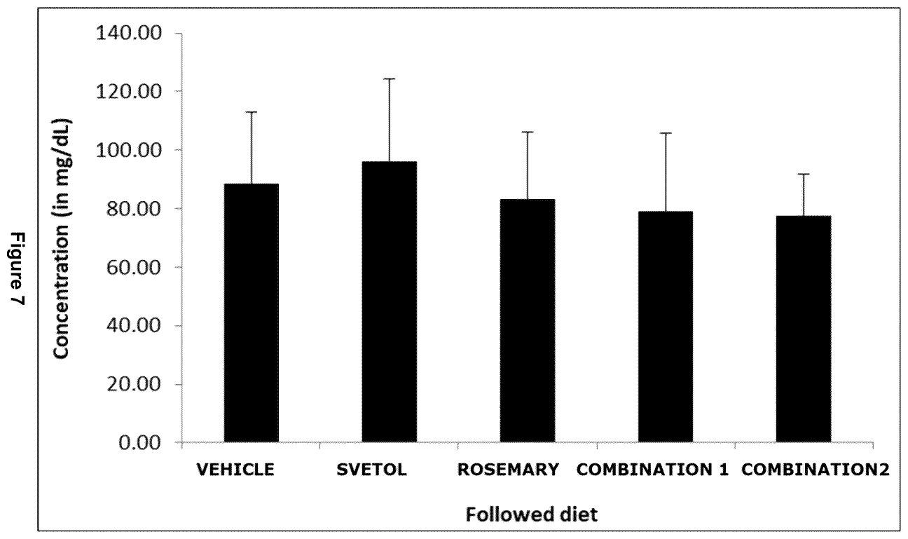

Table 10 and FIG. 7 depict the results obtained with respect to the determination of “Oxidized-LDL”.

TABLE 10
Individual oxidized LDL-Cholesterol concentration
obtained (in mg/dL) for each diet.

| | | | | COMBI- | COMBI- |
|---|---|---|---|---|---|
| | | | | NATION | NATION |
| | VEHICLE | SVETOL | ROSEMARY | DOSE 1 | DOSE 2 |
| | 78 | 100 | 97 | 76 | 73 |
| | 101 | 113 | 84 | 171 | 61 |
| | 105 | 84 | 71 | 68 | 97 |
| | 102 | 91 | 57 | 75 | 85 |
| | 114 | 71 | 77 | 80 | 55 |
| | 63 | 80 | 106 | 79 | 85 |
| | 51 | 50 | 105 | 79 | 57 |
| | 66 | 89 | 100 | 59 | 66 |
| | 126 | 75 | 53 | 63 | 74 |
| | 61 | 85 | 65 | 97 | 76 |
| | 58 | 165 | 81 | 63 | 71 |
| | 84 | 119 | 77 | 63 | 104 |
| | 88 | 65 | 62 | 109 | 88 |
| | 75 | 75 | 119 | 74 | 81 |
| | 114 | 144 | 49 | 54 | 97 |
| | 98 | 107 | 88 | 80 | 90 |
| | 125 | 115 | 124 | 75 | 62 |
| | No Sample | 103 | No Sample | 55 | 74 |
| Mean | 89 | 96 | 83 | 79 | 78 |
| SD | 24 | 28 | 23 | 27 | 14 |

A decrease of LDL-Cholesterol concentration of respectively 11% and 13% was only detected in both combination groups: COMBINATION DOSE 1 and DOSE 2. No significand decrease was observed in groups treated only with rosemary extract or only with green coffee extract,

These results clearly demonstrate a synergy between the rosemary extract and the green coffee extract.

Isoprostanes Determination

For each period, Tables 12 to 15 below show the obtained isoprostanes (PGF2) concentrations during the study.

TABLE 12
Obtained individual 8-iso-PGF2 α
concentrations for the Period I.1.

| Period | Product | Mouse # | PGF2 (pg/ml) | Mean | SD |
|---|---|---|---|---|---|
| I.1 | VEHICLE | 1 | 232 | 295 | 53 |
| | | 2 | 325 | | |
| | | 3 | 256 | | |
| | | 4 | 296 | | |
| | | 5 | 365 | | |
| | SVETOL | 6 | 214 | 201 | 35 |
| | | 7 | 250 | | |
| | | 8 | 156 | | |
| | | 9 | 182 | | |
| | | 10 | 205 | | |
| | ROSEMARY | 11 | 330 | 240 | 61 |
| | | 12 | 229 | | |
| | | 13 | 191 | | |
| | | 14 | 183 | | |
| | | 15 | 267 | | |
| | COMBINATION | 16 | 121 | 183 | 56 |
| | DOSE 1 | 17 | 179 | | |
| | | 18 | 274 | | |
| | | 19 | 176 | | |
| | | 20 | 166 | | |
| | COMBINATION | 21 | 291 | 263 | 59 |
| | DOSE 2 | 22 | 165 | | |
| | | 23 | 310 | | |
| | | 24 | 296 | | |
| | | 25 | 251 | | |

TABLE 13
Obtained individual 8-iso-PGF2 α
concentrations for the Period I.2.

| Period | Product | Mouse # | PGF2 (pg/ml) | Mean | SD |
|---|---|---|---|---|---|
| I.2 | COMBINATION | 26 | 395 | 285 | 144 |
| | DOSE 1 | 27 | 164 | | |
| | | 28 | 183 | | |
| | | 29 | 482 | | |
| | | 30 | 203 | | |
| | SVETOL | 31 | 273 | 276 | 94 |
| | | 32 | 264 | | |
| | | 33 | 420 | | |
| | | 34 | 268 | | |
| | | 35 | 157 | | |
| | COMBINATION | 36 | 470 | 319 | 105 |
| | DOSE 2 | 37 | 243 | | |
| | | 38 | 198 | | |
| | | 39 | 344 | | |
| | | 40 | 338 | | |
| | VEHICLE | 41 | 472 | 437 | 119 |
| | | 42 | 296 | | |
| | | 43 | 378 | | |
| | | 44 | 615 | | |
| | | 45 | 424 | | |
| | ROSEMARY | 46 | 333 | 294 | 100 |
| | | 47 | 397 | | |
| | | 48 | 129 | | |
| | | 49 | 294 | | |
| | | 50 | 315 | | |

TABLE 14
Obtained individual PGF2 concentrations for the Period II.

| Period | Product | Mouse # | PGF2 (pg/ml) | Mean | SD |
|---|---|---|---|---|---|
| II | ROSEMARY | 51 | 230 | 255 | 117 |
| | | 52 | 133 | | |
| | | 53 | 256 | | |
| | | 54 | 210 | | |
| | | 55 | 447 | | |
| | SVETOL | 56 | 267 | 195 | 49 |
| | | 57* | 174* | | |
| | | 58 | 151 | | |
| | | 59 | 222 | | |
| | | 60 | 161 | | |
| | VEHICLE | 61 | 226 | 201 | 51 |
| | | 62 | 121 | | |
| | | 63 | 191 | | |
| | | 64 | 211 | | |
| | | 65 | 257 | | |
| | COMBINATION | 66 | 302 | 203 | 56 |
| | DOSE 2 | 67 | 177 | | |
| | | 68 | 182 | | |
| | | 69 | 166 | | |
| | | 70 | 188 | | |
| | COMBINATION | 71 | 154 | 172 | 27 |
| | DOSE 1 | 72 | 210 | | |
| | | 73 | 181 | | |
| | | 74 | 178 | | |
| | | 75 | 139 | | |

*= Mouse with a tumour causing diluted blood.

TABLE 15
Obtained individual PGF2 concentrations for the Period III.

| Period | Treatment | Mouse # | PGF2 (pg/ml) | Mean | SD |
|---|---|---|---|---|---|
| III | ROSEMARY | 76 | 143 | 191 | 54 |
| | | 77 | 249 | | |
| | | 78 | 181 | | |
| | SVETOL | 79 | 209 | 186 | 27 |
| | | 80 | 157 | | |
| | | 81 | 190 | | |
| | VEHICLE | 82 | 222 | 296 | 64 |
| | | 83 | 329 | | |
| | | 84 | 337 | | |
| | COMBINATION | 85 | 185 | 197 | 12 |
| | DOSE 2 | 86 | 198 | | |
| | | 87 | 208 | | |
| | COMBINATION | 88 | 151 | 212 | 64 |
| | DOSE 1 | 89 | 204 | | |
| | | 90 | 280 | | |

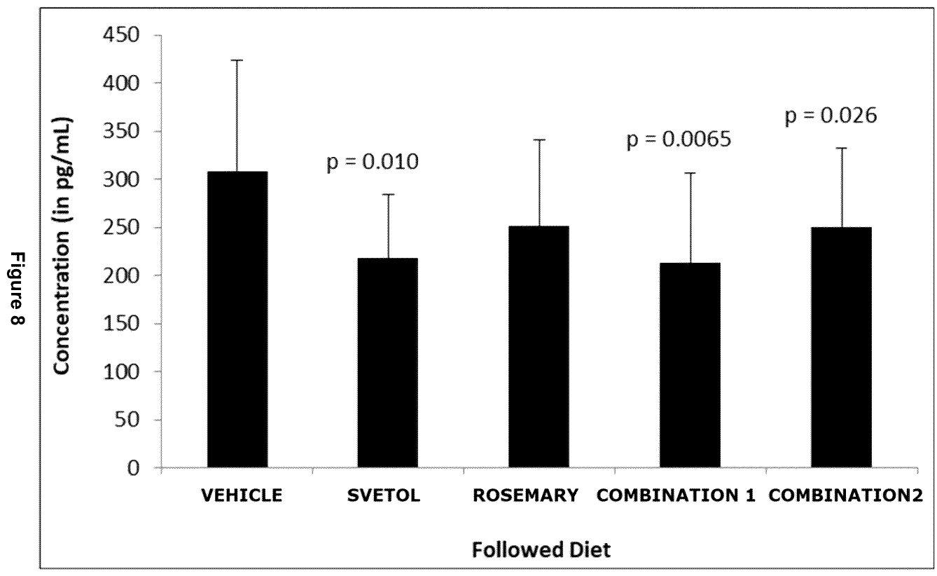

FIG. 8 shows the mean PGF2 values obtained for each group for all periods together. Statistical analysis shows that all the SVETOL products reduce significantly the isoprostanes levels in the mice's blood. COMBINATION DOSE 1 and COMBINATION DOSE 2 performed better than SVETOL alone. COMBINATION DOSE 2 provides a better effect even using lower doses as the individual extract alone: this is also synergy.

### CONCLUSION

The data indicate that the combination of green coffee extract and rosemary extract has a synergistic effect on HDL cholesterol (increase). Also there is a synergistic effect of the combination in the levels of oxidised LDL (decrease) and isoprostanes (decrease).

1. A combination comprising an extract obtained or obtainable from a plant of the Coffea genus and an extract obtained or obtainable from a plant of the Lamiaceae family, wherein the extract obtained or obtainable from a plant of the Lamiaceae family comprises:
a) from about 1%, 5%, 10%, 15%, 20%, 25%, 30%, 35%, 40% or 45% to about 95%, 90%, 85%, 80%, 75%, 70%, 65%, 60%, 55% or 50% by weight of the final composition (w/w) of carnosic acid, and/or
b) from about 1%, 5%, 10%, 15%, 20%, 25%, 30%, 35%, 40% or 45% to about 95%, 90%, 85%, 80%, 75%, 70%, 65%, 60%, 55% or 50% of carnosol by weight of the final composition (w/w).

2. A combination according to claim 1, wherein the extract obtained or obtainable from a plant of the Lamiaceae family comprises from about 15% to about 30% by weight of the extract of carnosic acid.

3. A combination according to claim 1, wherein the extract obtained or obtainable from a plant of the Lamiaceae family comprises from about 1% to about 5% by weight of the extract of carnosol.

4. A combination according to claim 1, wherein the extract obtained from a plant of the Lamiaceae family is an ethanol extract.

5. A combination according to claim 1, wherein the extract obtained or obtainable from a plant of the Coffea genus comprises from about 30% to about 60% by weight of the extract of chlorogenic acid.

6. A combination according to claim 1, wherein the extract obtained or obtainable from a plant of the Coffea genus is a hydro-alcoholic extract or a water extract.

7. A combination according to claim 1, wherein the extract obtained or obtainable from a plant of the Coffea genus is present in the composition in an amount of from 65% to 80% by weight of the composition and the extract obtained or obtainable from a plant of the Lamiaceae family is present in the composition in an amount of from 20% to 35% by weight of the composition.

8. A combination according to claim 1 that comprises
a) from about 19.5% to about 50% wt/wt, such as from about 30% to about 40% wt/wt, such as about 35% by weight of chlorogenic acid,
b) from about 3% to about 15% wt/wt, such as from about 3% to about 8%, such as about 5% wt/wt of carnosic acid, and
c) from about 0.2% to about 3% wt/wt, such as from about 0.4% to about 1% such as 0.6% wt/wt of carnosol.

9. A combination according to claim 1, wherein the extract obtained or obtainable from a plant of the Coffea genus is a green coffee bean extract and the extract of a plant of the Lamiaceae family is a rosemary leaf extract.

10. A mixture comprising
d) from about 19.5% to about 50% wt/wt, by weight of the composition of chlorogenic acid,
e) from about 3% to about 15% wt/wt by weight of the composition of carnosic acid, and
f) from about 0.2% to about 3% wt/wt, by weight of the composition of carnosol.

11. A mixture according to claim 10, wherein the chlorogenic acid is obtained from a plant of the Coffea genus and the carnosic acid and carnosol is obtained from a plant of the Lamiaceae family.

12. A combination according to claim 1 further comprising maltodextrin.

13. A pharmaceutical composition, a nutraceutical composition or a food composition comprising the combination according to claim 1 and optionally a physiologically acceptable excipient and/or carrier.

14. A combination according to claim 1 for use in preventing, alleviating and/or treating cardiovascular diseases and/or cerebrovascular diseases or for use in supporting cardiovascular and/or cerebrovascular health.

15. A combination according to claim 1 for use in reducing and/or preventing the formation of atherosclerotic plaques, LDL levels and/or isoprostanes levels and/or for use in increasing HDL levels.

16. A method of using a combination according to claim 1 in the manufacture of a medicament for use in preventing, alleviating and/or treating cardiovascular diseases and/or cerebrovascular diseases.

17. A method of using a combination according to claim 1 in the manufacture of a medicament for use in supporting cardiovascular and/or cerebrovascular health.

18. A method of using a combination according to claim 1 in the manufacture of a medicament for use in reducing the formation of atherosclerotic plaques and/or reducing LDL and/or increasing HDL, and/or decreasing isoprostanes.

19. A combination according to claim 1 for use in:
a) preventing, alleviating and/or treating cardiovascular diseases and/or cerebrovascular diseases, or
b) supporting cardiovascular and/or cerebrovascular health, or
c) reducing and/or preventing the formation of atherosclerotic plaques, reducing LDL levels, increasing HDL levels, and/or decreasing isoprostanes levels,
wherein the combination is administered in an amount to provide chlorogenic acid in an amount of from about 1 mg/kg to about 5 mg/kg, such as from about 1.5 mg/kg to about 3.5 mg/kg, such as about 1.6 mg/kg or about 3.22 mg/kg; and carnosol and carnosic acid in an amount of from about 0.1 mg/kg to about 2 mg/kg, such as from about 0.2 mg/kg to about 1 mg/kg, such as about 0.28 or about 0.55 mg/kg.

20. A combination according to claim 1 for use in:
a) preventing, alleviating and/or treating cardiovascular diseases and/or cerebrovascular diseases, or
b) supporting cardiovascular and/or cerebrovascular health, or
c) reducing and/or preventing the formation of atherosclerotic plaques, reducing LDL levels, increasing HDL levels, and/or decreasing isoprostanes levels,
wherein the combination, is administered in an amount to provide chlorogenic acid in an amount of from about 68 mg/day to about 250 mg/day, such as from about 95 mg/day to about 195 mg/day, such as about 97 mg/day or about 193 mg/day; and carnosol and carnosic acid in an amount of from about 10 mg/day to about 43 mg/day, such as from about 15 mg/day to about 35 mg/day, such as about 16.5 mg/day to about 33 mg/day.
